(19) United States
(12) Patent Application Publication
(10) Pub. No.: US 2011/0152303 A1
(43) Pub. Date: Jun. 23, 2011

(54) Chemical Compounds

(75) Inventor: Brian A Johns, Durham, NC (US)

(73) Assignee: GlaxoSmithKline LLC, Philadelphia, PA (US)

(21) Appl. No.: 12/970,366

(22) Filed: Dec. 16, 2010

Related U.S. Application Data
(60) Provisional application No. 61/287,789, filed on Dec. 18, 2009.

(51) Int. Cl. A61K 31/505; C07D 239/48; A61P 31/18

(52) U.S. Cl. 514/275; 544/323

(57) ABSTRACT

Novel diarylpyrimidine derivatives and pharmaceutically acceptable salts, solvates or hydrates thereof, designed to inhibit HIV reverse transcriptase, are provided, and a pharmaceutical composition containing the same, especially an anti-HIV agent. More specifically, novel diarylpyrimidine derivatives that are derivatives of the HIV reverse transcriptase inhibitor TMC278 of formula (1^{a}) are provided, and pharmaceutically acceptable salts, solvates or hydrates thereof.

This application is filed pursuant to 35 U.S.C. 111(a) as a United States Application which claims priority from U.S. Provisional Application Ser. No. 61/287,789 filed Dec. 18, 2009, the contents of which is hereby incorporated by reference.

### FIELD OF THE INVENTION

The present invention relates to novel diarylpyrimidine derivatives and pharmaceutically acceptable salts, solvates or hydrates thereof, and a pharmaceutical composition containing the same. The compositions are designed to inhibit human immunodeficiency virus (HIV), particularly HIV reverse transcriptase. They may thus be categorized as non-nucleoside reverse transcriptase inhibiters (NNRTIs). More specifically, embodiments of the invention relate to novel diarylpyrimidine derivatives that are derivatives of the HIV reverse transcriptase inhibitor TMC278, also known as rilpivirine.

### BACKGROUND OF THE INVENTION

It is known in the prior art that each element has unique number of protons in the atom's nucleus. The number of protons is called the atomic number and in a neutral state is equal to the number of electrons that surround the nucleus. The mass number however is defined as the number of protons and neutrons contained within the nucleus of a given atom. The molecular weight of an atom is equal to the number of protons and neutrons contained in that substance. Isotopes are defined as atoms that contain the same number of protons (which define the element) and different numbers of neutrons. Some isotopes are unstable and decay to give off radioactivity, while others are stable and do not decay. Hydrogen (H, 1H, protio) is the simplest atom containing only a single proton and no neutrons (atomic number 1, mass number 1). Deuterium (D, 2H, deutero) is a stable isotope of hydrogen that contains 1 proton and 1 neutron having an atomic number of 1 and a mass number of 2. Deuterium is present in a natural abundance of 0.015%.

Any carbon-hydrogen bond has a given energy resulting from vibrations in the molecule at a certain temperature. This energy is defined as the zero point energy. A corresponding carbon-deuterium bond also has a zero point energy under the same conditions; however, because of the greater mass of the deuterium (resulting from the extra neutron), the vibrations contribute to give a lower zero point energy relative to the hydrogen counterpart. The difference in the C—H and C-D bond energies result in a different activation energy requirement to reach an identical transition state. This difference in energy gives rise to what is called a kinetic isotope effect. This small but often significant energy difference can lead to retarded reaction rates in the deuterium analog when the bond is being broken in a rate limiting step during a chemical transformation. The approximate 1.2 kcal/mol energy difference that is often found when comparing the two isotope bonds (C—H versus C-D) can lead to a several fold reduction in reaction rate depending on the transition state dynamics. In some instances very little effect is noted between the bonds when the rate comparison is at or near 1. In other instances the rate of the hydrogen-containing reaction can be 7 times faster or greater, in certain circumstances, than the deuterium analog.

It is believed that this retardation of reaction rate for the C-D bond can potentially affect the metabolism of deuterated drug molecules in an in vitro or in vivo setting, thus rendering an altered pharmacokinetic profile of a drug molecule. Thus, one potential interest is to alter a drug substance by replacing hydrogen atoms with deuterium atoms. In addition, because the steric environment for the C-D and C—H bonds, among other properties, is identical, the potency and pharmacologic profile of a deuterated compound are expected to be the same as the parent drug substance, when the molecule's mechanism of action does not involve a carbon-hydrogen (or carbon-deuterium) bond breaking event.

Among viruses, human immunodeficiency virus (HIV), a kind of retrovirus, is known to cause acquired immunodeficiency syndrome (AIDS). The therapeutic agent for AIDS is mainly selected from a group of reverse transcriptase inhibitors (e.g., AZT, 3TC) and protease inhibitors (e.g., Indinavir), but they are proved to be accompanied by side effects such as nephropathy and the emergence of resistant viruses. Thus, the development of anti-HIV agents having the other mechanism of action has been desired.

On the other hand, a combination therapy is reported to be efficient in treatment for AIDS because of the frequent emergence of the resistant mutant. Reverse transcriptase inhibitors and protease inhibitors are clinically used as anti-HIV agents; however, agents having the same mechanism of action often exhibit cross-resistance or only an additional activity. Therefore, anti-HIV agents that do not exhibit cross-resistance, or that exhibit synergistic activity with an additional agent, are desired.

One such anti-HIV agent is TMC278 or rilpivirine (CAS name: 4-([4-([4-([1E]-2-cyanoethenyl)-2,6-dimethylphenyl]-amino)-2-pyrimidinyl]-amino)-benzonitrile), an HIV reverse transcriptase inhibitor useful in the treatment of HIV-related conditions such as AIDS. Rilpivirine is a diarylpyrimidine (DAPY) ‘next generation’ NNRTI, with molecular formula C₂₂H₁₈N₆ and molecular mass 366.42 Daltons. TMC278/rilpivirine is an investigational NNRTI with a high genetic barrier to the development of resistance, having strong binding properties and conformational flexibility that allows it to overcome mutations conferring NNRTI resistance. TMC278 has exhibited promising results in a number of phase I and phase II clinical trials. Embodiments of the present invention can increase metabolic stability by advantageously employing the deuterium isotope effect in the design and preparation of deuterium analogs of TMC278. Embodiments of the invention will allow for reduction in pk variability, increase in exposure or half-life, and/or reduction in the amount of drug substance required to achieve efficacy.

### SUMMARY OF THE INVENTION

Embodiments of the present invention provide deuterated compounds and uses thereof as reverse transcriptase inhibitors. Such compounds may be useful in the treatment of conditions related to infection with the human immunodeficiency virus (HIV).

In a first embodiment of the invention there is provided a compound of Formula (I):

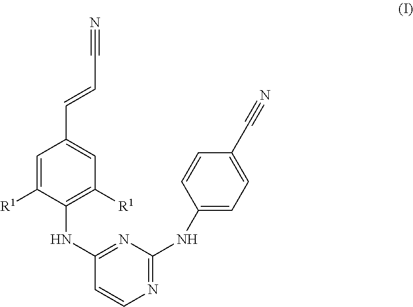

wherein:

each R¹ is independently selected from -D or —H, where at least one R¹ substituent is -D, or a salt, solvate or hydrate thereof.

In a second aspect of the present invention, there is provided a compound of formula (I)

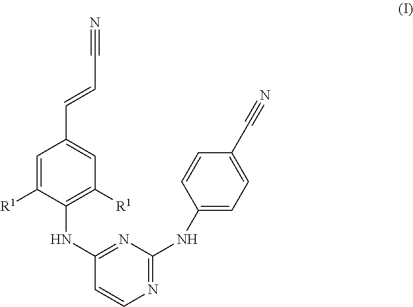

wherein:

each R¹ is independently selected from -D or —H, wherein the level of deuterium incorporation in each R¹ group substituted with -D is at least 50%;
or a salt, solvate or hydrate thereof.

In a third aspect of the present invention, there is provided a pharmaceutical composition comprising a therapeutically effective amount of a compound of formula (I) or a pharmaceutically acceptable salt, solvate or hydrate thereof and one or more of pharmaceutically acceptable carriers, diluents and excipients.

In a fourth aspect of the present invention, there is provided a method of treating a virus susceptible to HIV reverse transcriptase inhibition in a mammal, comprising administering to said mammal a therapeutically effective amount of a compound of formula (I) or a pharmaceutically acceptable salt, solvate or hydrate thereof.

In a fifth aspect of the present invention, there is provided a method of inhibiting HIV related reverse transcriptase in a mammal, comprising administering to said mammal a therapeutically effective amount of a compound of formula (I) or a pharmaceutically acceptable salt, solvate or hydrate thereof.

In a sixth aspect of the present invention, there is provided a method of treating reverse transcriptase resistant HIV in a mammal, comprising administering to said mammal a therapeutically effective amount of a compound of formula (I) or a pharmaceutically acceptable salt, solvate or hydrate thereof.

In a seventh aspect of the present invention, there is provided a method of preventing development of reverse transcriptase resistant HIV in a mammal, comprising administering to said mammal a therapeutically effective amount of a compound of formula (I) or a pharmaceutically acceptable salt, solvate or hydrate thereof.

One particular embodiment of the invention provides a method of preventing development of reverse transcriptase inhibitor-resistant HIV in a mammal, comprising administering to the mammal a therapeutically effective amount of a compound of formula (I) or a pharmaceutically acceptable salt, solvate or hydrate thereof, wherein the reverse transcriptase inhibitor against which HIV resistance is prevented is not a chemically or enzymatically-deuterated reverse transcriptase inhibitor.

Another embodiment provides a method of treating a virus susceptible to HIV reverse transcriptase inhibition in a mammal comprising administering to said mammal a long-acting parenteral composition, wherein the long-acting parenteral composition comprises a therapeutically effective amount of compound of formula (1), as described above, or a pharmaceutically acceptable salt, solvate or hydrate thereof. In related embodiments, the long-acting parenteral composition further comprises at least one additional agent useful in treating HIV infection, or at least one additional agent useful as a pharmacological enhancer.

Still another embodiment of the invention provides a method of treating reverse transcriptase inhibitor-resistant HIV in a mammal comprising administering to said mammal a long-acting parenteral composition, wherein the long-acting parenteral composition comprises a therapeutically effective amount of compound of formula (1), as described above, or a pharmaceutically acceptable salt, solvate or hydrate thereof. In related embodiments, the long-acting parenteral composition further comprises at least one additional agent useful in treating HIV infection or at least one additional agent useful as a pharmacological enhancer.

Another embodiment of the invention provides a method of inhibiting HIV-related reverse transcriptase in a mammal comprising: administering to said mammal a long-acting parenteral composition, wherein the long-acting parenteral composition comprises a therapeutically effective amount of a compound of formula (1), as described above, or a pharmaceutically acceptable salt, solvate or hydrate thereof. In related embodiments, the long-acting parenteral composition further comprises at least one additional agent useful in treating HIV infection, or at least one additional agent useful as a pharmacological enhancer. In another related embodiment, the methods described further comprise administering at least one additional agent useful in treating HIV infection, and/or further comprising administering at least one additional agent useful as a pharmacological enhancer. In a related embodiment, said methods may further comprise administering at least one additional agent useful in treating HIV infection, with or without a pharmaceutical enhancer.

Yet another embodiment provides a compound selected from the group consisting of:
4-([4-([4-([1E]-2-cyanoethenyl)-2,6-dimethylphenyl]-amino)-2-pyrimidinyl]-amino)-benzonitrile-d₃,

whose structure is:

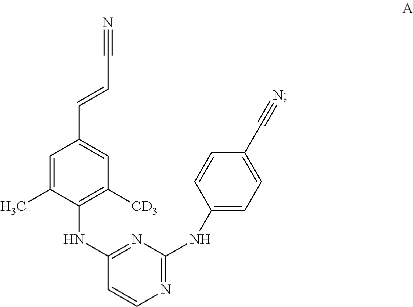

4-([4-([4-([1E]-2-cyanoethenyl)-2,6-dimethylphenyl]-amino)-2-pyrimidinyl]-amino)-benzonitrile-d₄,

whose structure is:

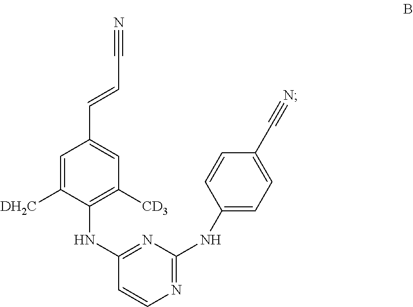

4-([4-([4-([1E]-2-cyanoethenyl)-2,6-dimethylphenyl]-amino)-2-pyrimidinyl]-amino)-benzonitrile-d₅,

whose structure is:

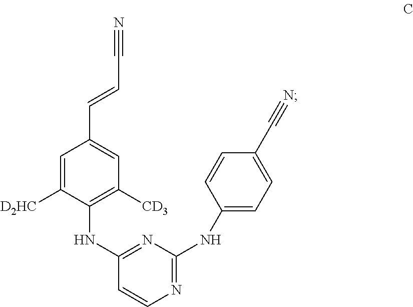

4-([4-([4-([1E]-2-cyanoethenyl)-2,6-dimethylphenyl]-amino)-2-pyrimidinyl]-amino)-benzonitrile-d₆,

whose structure is:

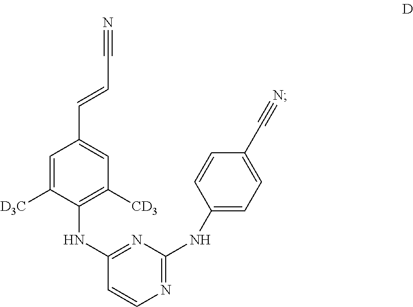

4-([4-([4-([1E]-2-cyanoethenyl)-2,6-dimethylphenyl]-amino)-2-pyrimidinyl]-amino)-benzonitrile-d₂,

whose structure is:

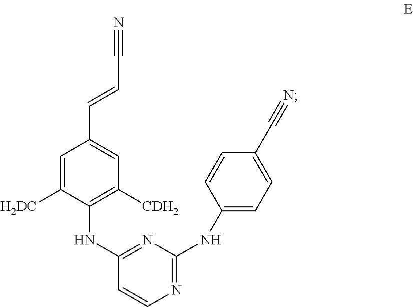

4-([4-([4-([1E]-2-cyanoethenyl)-2,6-dimethylphenyl]-amino)-2-pyrimidinyl]-amino)-benzonitrile-d₃,

whose structure is:

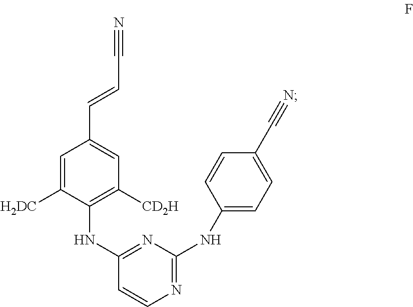

4-([4-([4-([1E]-2-cyanoethenyl)-2,6-dimethylphenyl]amino)-2-pyrimidinyl]-amino)-benzonitrile-d₄,

whose structure is:

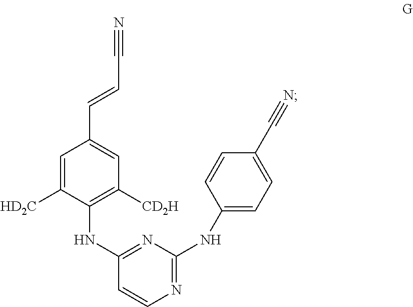

4-([4-([4-([1E]-2-cyanoethenyl)-2,6-dimethylphenyl]-amino)-2-pyrimidinyl]-amino)-benzonitrile-d₂,

whose structure is:

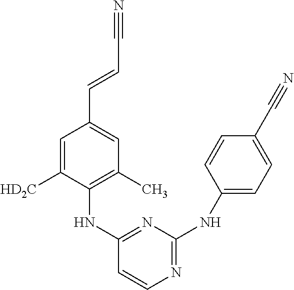

H; and
4-([4-([4-([1E]-2-cyanoethenyl)-2,6-dimethylphenyl]-amino)-2-pyrimidinyl]-amino)-benzonitrile-d₁,

whose structure is:

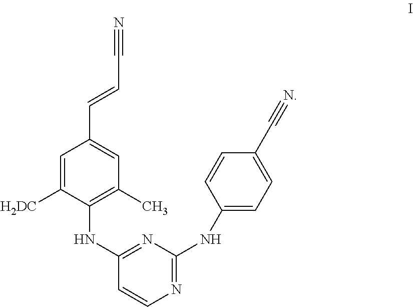

In a particular embodiment, there is provided a compound which is 4-([4-([4-([1E]-2-cyanoethenyl)-2,6-dimethylphenyl]-amino)-2-pyrimidinyl]-amino)-benzonitrile-d₆, and in a still more particular embodiment, the compound has the structure:

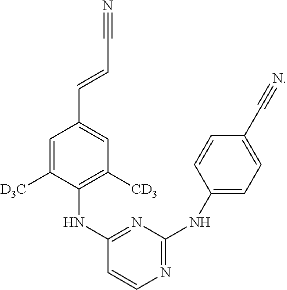

In related embodiments there is provided a long-acting parenteral pharmaceutical composition comprising a therapeutically effective amount of a compound of formula (I), or a pharmaceutically acceptable salt, solvate or hydrate thereof, and one or more of a pharmaceutically acceptable carrier, diluent or excipient. Particular related embodiments provide such a composition, wherein the long-acting parenteral pharmaceutical composition further comprises at least one additional agent useful in treating HIV infection; or at least two additional agents useful in treating HIV infection. Still more related particular embodiments provide a long-acting parenteral pharmaceutical composition as described, wherein the at least one additional agent is selected from the group consisting of GSK1349572, GSK1265744 and GSK1247303. Other related particular embodiments provide such a long-acting parenteral pharmaceutical composition, wherein the at least one additional agent IDX-899. And still other related particular embodiments provide a long-acting parenteral pharmaceutical composition as described, wherein the at least one additional agent is selected from the group consisting of GSK1349572, GSK1265744 and GSK1247303, and wherein the composition further comprises IDX-899.

### DETAILED DESCRIPTION OF SPECIFIC EMBODIMENTS

Definitions. As used in this description and the accompanying claims, the following terms shall have the meanings indicated, unless the context otherwise requires:

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “HIV” refers to the human immunodeficiency virus. HIV is the causative agent for AIDS.

As used herein the term “AIDS” refers to acquired immunodeficiency syndrome. AIDS is a disease characterized by destruction of the immune system, particularly of CD4+ T-cells, with attendant susceptibility to opportunistic infections.

As used herein the term “HIV-related reverse transcriptase” means an reverse transcriptase from any variant of a human immunodeficiency virus (HIV), an reverse transcriptase from a human T-cell lymphotropic virus (“HTLV”), or an reverse transcriptase from any related immunodeficiency virus which targets a non-human mammal, such as an reverse transcriptase from a feline immunodeficiency virus (“FIV”), an reverse transcriptase from a feline leukemia virus (“FLV”), an reverse transcriptase from a simian immunodeficiency virus (“SIV”), an reverse transcriptase from a bovine leukemia virus (“BLV”), and reverse transcriptase from an equine infectious anemia virus (“EIAV”), and other such reverse transcriptases. HIV-related reverse transcriptase includes, but is not limited to, an reverse transcriptase from HTLV-1, HTLV-2, HTLV-3, HTLV-4, HIV-1 (formerly known as HTLV-3), HIV-2, FIV, FIV IV, BLV, MLV (murine leukemia virus), and simian viruses such as SRV1-D. SRV2-D, SMP-D, sooty mangabey SIVSMMH4, macaque SIVMM142, macaque SIVMM251, African green monkey STLV-Illagm, and MoMLV (Moloney murine leukemia virus).

As used herein, a “reverse transcriptase inhibitor that is not a chemically- or enzymatically-deuterated reverse transcriptase inhibitor” means any reverse transcriptase inhibitor comprising a deuterium level within ±5% that which would be present based on the natural abundance for the deuterium isotope. An reverse transcriptase inhibitor that is not a chemically- or enzymatically-deuterated reverse transcriptase inhibitor encompasses natural and synthetically prepared reverse transcriptase inhibitors that have not had any of the hydrogens of the reverse transcriptase inhibitor enriched for the deuterium isotope, whether by chemical, enzymatic, isotope exchange, or other means, such growing an reverse transcriptase inhibitor-producing organism in or on a deuterium enriched medium, or in/on a medium comprising nutrients, carbon sources and other media components that have been enriched with deuterium, and then isolating the resulting reverse transcriptase inhibitors having enriched deuterium levels, and the like.

As used herein, the term “D” refers to a deuterium atom. Deuterium (D, 2H, deutero) is a stable isotope of hydrogen that contains 1 proton and 1 neutron having an atomic number of 1 and a mass number of 2. Deuterium is present in a natural abundance of 0.015%.

TMC278 is an HIV-1 reverse transcriptase inhibitor having the structure of a compound of formula (I) wherein all of the R¹ groups are —H. Thus, TMC278 is a compound of formula (I^{a}):

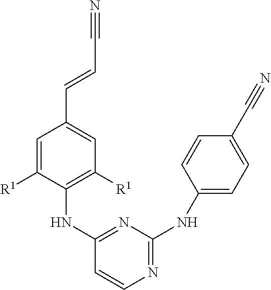

(I^{a})

wherein R¹ is CH₃.

As used herein, the term “deuterated TMC278” or “rilpivirine” means TMC278 or rilpivirine wherein at least one of the hydrogens is substituted with deuterium. That is, a compound of formula (I) wherein at least one R¹ is a deuterium.

As used herein, the term “enriched deuterated TMC278 or rilpivirine” means TMC278 or rilpivirine wherein in each hydrogen substituted with deuterium the deuterium is incorporated at a level of at least 50%. That is, a compound of formula (I) wherein the level of incorporation of deuterium in each hydrogen substituted with deuterium is at least 50%.

As used herein and known in the art, the term “H” refers to a hydrogen atom. Hydrogen (H, 1H) is a stable isotope of hydrogen that contains 1 proton having an atomic number of 1 and a mass number of 1. Also, unless otherwise stated when a particular R¹ is substituted with —H it is understood to have hydrogen and deuterium at their natural abundance isotopic composition.

It will be apparent to one skilled in the art that due to the natural abundance of deuterium (0.015%) that compounds of formula I will be present at some minor level in compounds previously made wherein all positions were dictated to be ‘hydrogens’. However the low percentage of deuterium isotopes in mono deuterium containing analogs and even lower (0.015%×0.015%) for dual deuterium containing analogs and henceforth higher deuteron analogs is insignificant to the pharmacological effect of previously described drug molecules.

In the present invention, the compounds of formula I are substantially enriched in deuterium to levels of 50% at the indicated positions. In many cases the preferred level of deuterium incorporation at the indicated position is above 75%, 90%, 95% or 98% and approaching limits of quantitation for determining hydrogen (¹H) content. It will also be understood that compounds with multiple deuterium atoms incorporated will have isotopic mixtures dependent on the level of incorporation at each position.

Accordingly, in one embodiment, the level of deuterium incorporation in each R¹ group of the compound of formula (I) indicated as substituted with deuterium is at least 50%. In another embodiment, the level of deuterium incorporation in each R¹ group of the compound of formula (I) indicated as substituted with deuterium is at least 75%. In still another embodiment, the level of deuterium incorporation in each R¹ group of the compound of formula (I) indicated as substituted with deuterium is at least 90%. In a further embodiment, the level of deuterium incorporation in each R¹ group in the compound of formula (I) indicated as substituted with deuterium is at least 95%. In another embodiment, the level of deuterium incorporation in each R¹ group of the compound of formula (I) indicated as substituted with deuterium is at least 98%. In a further embodiment, the level of deuterium incorporation in each R¹ group of the compound of formula (I) indicated as substituted with deuterium approaches 100% and is limited by the limits of quantitation for detecting hydrogen content.

In one embodiment, the level of deuterium incorporation in each R¹ group of the compound of formula (I) indicated as substituted with deuterium is at least 20%. In another embodiment, the level of deuterium incorporation in each R¹ group of the compound of formula (I) indicated as substituted with deuterium is at least 25%. In still another embodiment, the level of deuterium incorporation in each R¹ group of the compound of formula (I) indicated as substituted with deuterium is at least 30%. In a further embodiment, the level of deuterium incorporation in each R¹ group in the compound of formula (I) indicated as substituted with deuterium is at least 35%. In another embodiment, the level of deuterium incorporation in each R¹ group of the compound of formula (I) indicated as substituted with deuterium is at least 40%. In a further embodiment, the level of deuterium incorporation in each R¹ group of the compound of formula (I) indicated as substituted with deuterium is at least 45%.

In one embodiment, the level of deuterium incorporation in each R¹ group of the compound of formula (I) indicated as substituted with deuterium is greater than 0.015%. In another embodiment, the level of deuterium incorporation in each R¹ group of the compound of formula (I) indicated as substituted with deuterium is at least 1%. In still another embodiment, the level of deuterium incorporation in each R¹ group of the compound of formula (I) indicated as substituted with deuterium is at least 3%. In a further embodiment, the level of deuterium incorporation in each R¹ group in the compound of formula (I) indicated as substituted with deuterium is at least 5%. In another embodiment, the level of deuterium incorporation in each R¹ group of the compound of formula (I) indicated as substituted with deuterium is at least 10%. In a further embodiment, the level of deuterium incorporation in each R¹ group of the compound of formula (I) indicated as substituted with deuterium is at least 15%.

It will be understood by those skilled in the art that the level of deuterium incorporation in each R¹ group indicated as substituted with deuterium may be the same or similar or may be different with the only limitation being the recited lower limit. For instance when the lower limit is at least 50% incorporation of deuterium, each deuterium substituted for hydrogen may be incorporated at any value of 50% or greater. That is, one deuterium substituted R¹ may have 60% deuterium incorporation whereas another deuterium substituted R¹ may have 80% deuterium incorporation and so on for other deuterium substituted R¹ groups.

Certain of the compounds described herein contain one or more chiral centers, or may otherwise be capable of existing as multiple stereoisomers. The scope of the present invention includes mixtures of stereoisomers as well as purified enantiomers or enantiomerically and/or diastereomerically enriched mixtures. Also included within the scope of the invention are the individual isomers of the compounds of the present invention, as well as any wholly or partially equilibrated mixtures thereof. The present invention also includes the individual isomers of the compounds represented by the formulas above as mixtures with isomers thereof in which one or more chiral centers are inverted. Also, it is understood that any tautomers and mixtures of tautomers of the compounds of formula (I) are included within the scope of the compounds of formula (I).

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise base addition salts derived from a removal of the hydroxyl group hydrogen on a compounds of formula (I). Salts derived from appropriate bases include alkali metal (e.g. sodium), alkaline earth metal (e.g., magnesium), ammonium, NW₄⁺ (wherein W is C₁₋₄ alkyl) and other amine salts. Physiologically acceptable salts of a compound with a hydroxy group include the anion of said compound in combination with a suitable cation such as Na⁺, NH₄⁺, and NW₄⁺ (wherein W is a C₁₋₄alkyl group). Preferred salts include sodium, calcium, potassium, magnesium, choline, meglumine, hydrochloride, and quaternary ammonium. Other salts, which are not pharmaceutically acceptable, may be useful in the preparation of compounds of this invention and these form a further aspect of the invention.

While it is possible that, for use in therapy, therapeutically effective amounts of a compound of formula (I), as well as salts, solvates or hydrates thereof, may be administered as the raw chemical, it is possible to present the active ingredient as a pharmaceutical composition. Accordingly, the invention further provides pharmaceutical compositions, which include therapeutically effective amounts of the compound of formula (I) or salts, solvates or hydrates thereof, and one or more pharmaceutically acceptable carriers, diluents, or excipients. The compounds of the formula (I) or salt, solvate or hydrate thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salt, solvate or hydrate thereof, with one or more pharmaceutically acceptable carriers, diluents or excipients.

Pharmaceutical formulations may be presented in unit dose forms containing a predetermined amount of active ingredient per unit dose. Such a unit may contain, for example, 100 mg to 5 g, 0.5 mg to 1 g, 1 mg to 700 mg, or 5 mg to 100 mg of a compound of the formula (I), depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

The present compound can be administered orally or parenterally. In the case of oral administration, the present compound can be also used as a conventional preparation, for example, as any dosage form of a solid agent such as tablets, powders, granules, capsules and the like; an aqueous agent; an oily suspension; or a liquid agent such as syrup and elixir. In the case of parenteral administration, the present compound can be used as an aqueous or oily suspension injectable, or a nasal drop. Upon preparation of it, conventional excipients, binders, lubricants, aqueous solvents, oily solvents, emulsifiers, suspending agents, preservatives, stabilizers and the like may be arbitrarily used. As an anti-HIV-drug, particularly, an oral agent is preferable. A preparation of the present invention is prepared by combining (e.g. mixing) a therapeutically effective amount of the present compound with a pharmaceutically acceptable carrier or diluent.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solutions, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) or salts, solvates or hydrates thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) or salts, solvates or hydrates thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

In one embodiment, the pharmaceutical formulation containing a compound of formula I or a salt, solvate or hydrate thereof is a formulation adapted for parenteral administration. In another embodiment, the formulation is a long-acting parenteral formulation. In a further embodiment, the formulation is a nano-particle formulation.

A therapeutically effective amount of a compound of the present invention will depend upon a number of factors including, for example, the age and weight of the human or other animal, the precise condition requiring treatment and its severity, the nature of the formulation, and the route of administration, and will ultimately be at the discretion of the attendant physician or veterinarian. An effective amount of a salt or hydrate thereof, may be determined as a proportion of the effective amount of the compound of formula (I) or salts, solvates or hydrates thereof per se.

The compounds of formula (I) or salts, solvates or hydrates thereof are believed to have activity in stopping or reducing the effects of HIV as a result of inhibition of HIV-1 reverse transcriptase.

Accordingly, there is provided a method of treating a virus susceptible to reverse transcriptase inhibition in a mammal, including administering to said mammal a therapeutically effective amount of a compound of formula (I) or a pharmaceutically acceptable salt, solvate or hydrate thereof. In one embodiment, the virus is HIV. In another embodiment, the reverse transcriptase is HIV-1 reverse transcriptase. In one embodiment, the mammal is a human.

In another aspect of the present invention, there is provided a method of inhibiting HIV reverse transcriptase in a mammal, including administering to said mammal a therapeutically effective amount of a compound of formula (I) or a pharmaceutically acceptable salt, solvate or hydrate thereof. In one embodiment, the mammal is a human.

In another aspect of the present invention, there is provided a method of treating reverse transcriptase inhibitor resistant HIV in a mammal, comprising administering to said mammal a therapeutically effective amount of a compound of formula (I) or a pharmaceutically acceptable salt, solvate or hydrate thereof. In one embodiment, the mammal is a human.

In another aspect of the present invention, there is provided a method of preventing development of reverse transcriptase inhibitor resistant HIV in a mammal, comprising administering to said mammal a therapeutically effective amount of a compound of formula (I) or a pharmaceutically acceptable salt, solvate or hydrate thereof. In one embodiment, the mammal is a human.

The compounds of the present invention and their salts, solvates, hydrates or other pharmaceutically acceptable derivatives thereof, may be employed alone or in combination with other therapeutic agents. The compounds of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound of the present invention and salts, solvates, hydrates or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The amounts of the compound(s) of formula (I) or salts, solvates or hydrates thereof and the other pharmaceutically active agent(s) and the relative timings of administration will be selected in order to achieve the desired combined therapeutic effect.

The present invention may be used in combination with one or more agents useful in the prevention or treatment of HIV. Examples of such agents include:
Nucleoside reverse transcriptase inhibitors such as zidovudine, didanosine, lamivudine, zalcitabine, abacavir, stavidine, adefovir, adefovir dipivoxil, fozivudine, todoxil, emtricitabine, alovudine, amdoxovir, elvucitabine, and similar agents;
Non-nucleoside reverse transcriptase inhibitors (including an agent having anti-oxidation activity such as immunocal, oltipraz, etc.) such as nevirapine, delavirdine, efavirenz, loviride, immunocal, oltipraz, capravirine, TMC-125, and similar agents;
Protease inhibitors such as saquinavir, ritonavir, indinavir, nelfinavir, amprenavir, fosamprenavir, brecanavir, darunavir, atazanavir, tipranavir, palinavir, lasinavir, and similar agents;
Entry inhibitors such as enfuvirtide (T-20), T-1249, PRO-542, PRO-140, TNX-355, BMS-806, 5-Helix and similar agents;
Integrase inhibitors such as L-870,810, MK-0518, Raltegravir, GS-9137, JTK-303, elvitegravir, GSK1349572, GSK1265744 and similar agents;
Budding inhibitors such as PA-344 and PA-457, and similar agents; and CXCR4 and/or CCR5 inhibitors such as vicriviroc (Sch-C), Sch-D, TAK779, maraviroc (UK 427,857), TAK449, as well as those disclosed in WO 02/74769, PCT/US03/39644, PCT/US03/39975, PCT/US03/39619, PCT/US03/39618, PCT/US03/39740, and PCT/US03/39732, and similar agents.

The compounds of the present invention and their salts, solvates, hydrates or other pharmaceutically acceptable derivatives thereof, may be provided as a long-acting parenteral pharmaceutical composition comprising a therapeutically effective amount of a compound of formula (I), or a pharmaceutically acceptable salt, solvate or hydrate thereof, and one or more of pharmaceutically acceptable carriers, diluents and excipients and at least one additional agent useful in treating HIV infection; or at least two additional agents useful in treating HIV infection. Such long-acting parenteral pharmaceutical compositions may also comprise at least one additional agent selected from the group consisting of GSK1349572, GSK1265744 and GSK1247303. Other long-acting parenteral pharmaceutical compositions may comprise at least one additional agent wherein the at least one additional agent is IDX-899. Related pharmaceutical compositions of the invention may provide a long-acting parenteral pharmaceutical composition comprising a therapeutically effective amount of a compound of formula (I), one additional agent selected from the group consisting of GSK1349572, GSK1265744 and GSK1247303, and a second agent, IDX-899.

The scope of combinations of compounds of this invention with HIV agents is not limited to those mentioned above, but includes in principle any combination with any pharmaceutical composition useful for the treatment of HIV. As noted, in such combinations the compounds of the present invention and other HIV agents may be administered separately or in conjunction. In addition, one agent may be administered prior to, concurrent to, or subsequent to the administration of other agent(s).

The compounds of this invention may be made by a variety of methods, including standard chemistry. Any previously defined variable will continue to have the previously defined meaning unless otherwise indicated. Illustrative examples are set out below in the Schemes 1-6 below.

Certain embodiments of the present invention will now be illustrated by way of example only. The physical data obtained for the compounds exemplified is consistent with the assigned structure of those compounds.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

g (grams); mg (milligrams);
L (liters); mL (milliliters);
μL (microliters); psi (pounds per square inch);
M (molar); mM (millimolar);
i. v. (intravenous); Hz (Hertz);
MHz (megahertz); mol (moles);
mmol (millimoles); rt (room temperature);
min (minutes); h (hours);
mp (melting point); TLC (thin layer chromatography);
Tᵣ (retention time); RP (reverse phase);
eq (equivalents); nM (nanomolar);

Unless otherwise indicated, all temperatures are expressed in ° C. (degrees Centigrade). All reactions are conducted under an inert atmosphere at room temperature unless otherwise noted. All reference to brine refers to a saturated aqueous solution of NaCl.

¹H NMR spectra were recorded on a Varian INOVA-400 (400 MHz) or a Bruker Avance 3 (400 or 500 MHz) spectrometer. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), br (broad).

Several synthetic approaches to preparing TMC278, deuterated TMC278, and enriched deuterated TMC278 are illustrated in Schemes 1-5 following. It will be apparent to one skilled in the art that several typical reagents or compounds employed in this synthetic route can be modified to include deuterium atoms and thus this general route can be modified to produce compounds of formula I.

Of particular note are published patent applications WO1999/50250A1, WO2004/016581A1 and WO2006/125809A1 covering the parent structure (I^{a}), incorporated by reference herein. In some cases it may be more convenient to synthesize a compound of formula (I) as a racemic mixture and separate the enantiomers using methods known to one skilled in the art.

One synthetic approach to TMC278 (formula (1^{a}), an analogue of formula (I) comprising no synthetically added deuterium atoms, is shown in scheme 1 wherein TMC278 is the final product. It will be apparent to one skilled in the art that several typical reagents or compounds employed in this synthetic route can be modified to include deuterium atoms and thus this general route can be modified to produce compounds of formula I.

Condensation of the known guanidine 1 with the known electrophile 2 to give the 4-hydroxy pyrimidine 3 is demonstrated in WO2006/125809. Treatment of this intermediate with POCl₃ and the like can be used to form a compound such as 4 as shown in WO2004/016581. This material can be condensed with aniline 6 which may be prepared via known methods in the aforementioned reference to give a compound such as

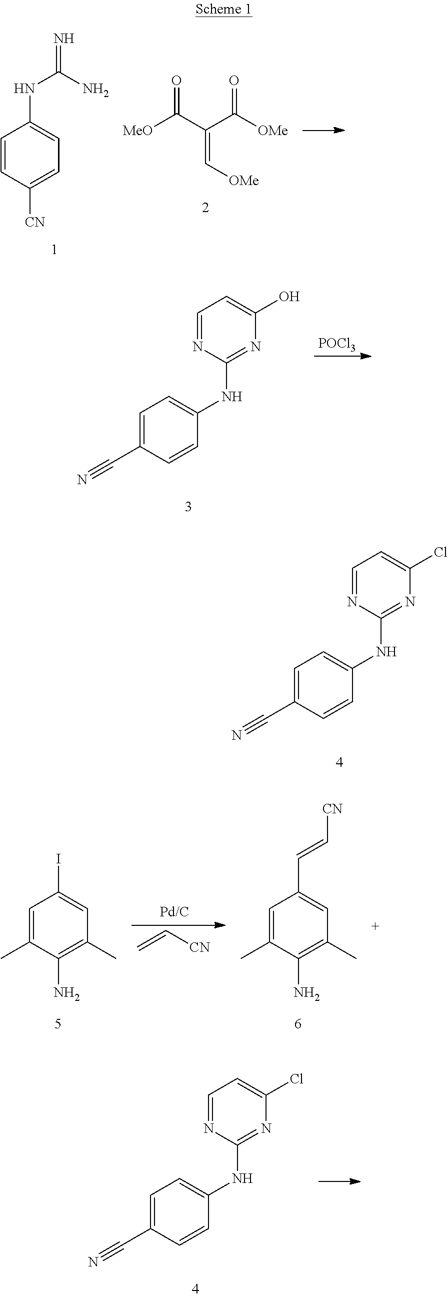

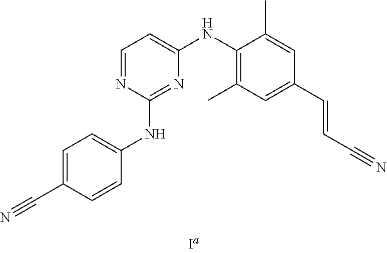

One particularly useful modification of scheme 1 is shown in scheme 2 whereby R¹ may consist of deuterium substitution as outlined in the general formula. Heck coupling of a compound such as 7 under similar conditions using a source of Pd(0) and acrylonitrile will provide a compound such as 8. Condensation with the 4-chloropyrimidine 4 can serve to provide a compound of formula I.

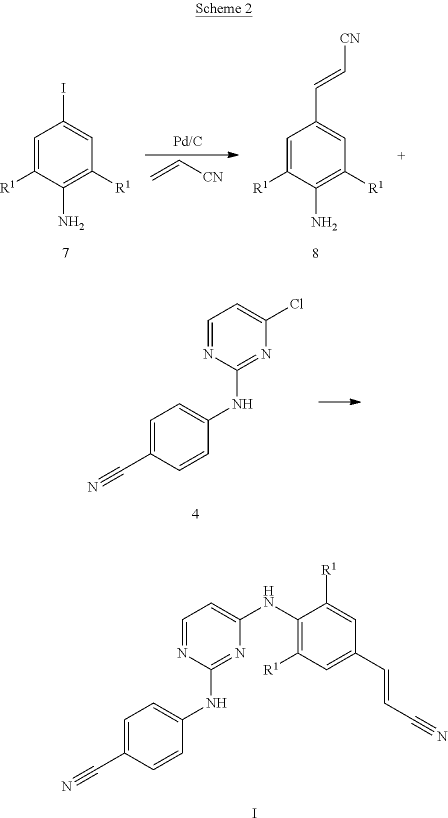

In order to prepare a compound such as 7 several methods to install the deuterium atoms may be considered. However it may be preferred to install the deuterium prior to halogenation of the aniline such that a transformation such as that shown in scheme 3 is of particular use. It is known that iodination of a 2,6-dimethylaniline can be achieved using reactive iodination reagents such as iodine monochloride. This can be found in Tetrahedron 2005, 61(49), 11657-11663 wherein R¹ is H by way of example.

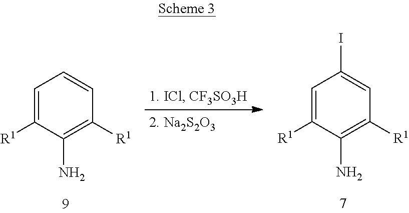

One possible method of making a compound of formula 9 is a deuterium exchange method whereby a known compound such as 10 can be treated with base under thermodynamic conditions to form a deuterated derivative such as 9^{a}. Conditions may include but are not limited to bases such as NaOD, NaOCD₃, Et₃N, pyridine, DBU and the like (scheme 4). In addition several methods are available in the literature as well as a compound such as 9^{a} is available from commercial sources.

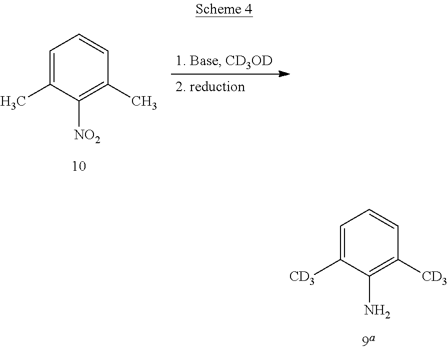

A similar method using kinetic control can be employed to make a compound such as 9^{b}. In this case a single deprotonation is performed with a base in a controlled stoichiometry such as LDA followed by a quench with a deuterium source for protonation. In this case a mono-deutero derivative such as 9^{b} may result (scheme 5).

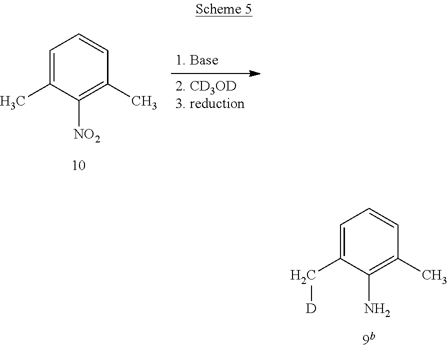

In a similar manner to the methods described above, the d₆ containing compound of formula I can be made as shown in scheme 6. Deuterium exchange may be accomplished using a heterogeneous catalyst (Pd/C) in the presence of deuterium oxide and deuterated methanol to give a compound such as 9′. Iodination as described above may provide a compound such as 7^{a} which can be treated with acrylonitrile to perform a Heck cross-coupling to give 8^{a}. Condensation of 8^{a} and 4 can be used to form a compound of formula I^{a}.

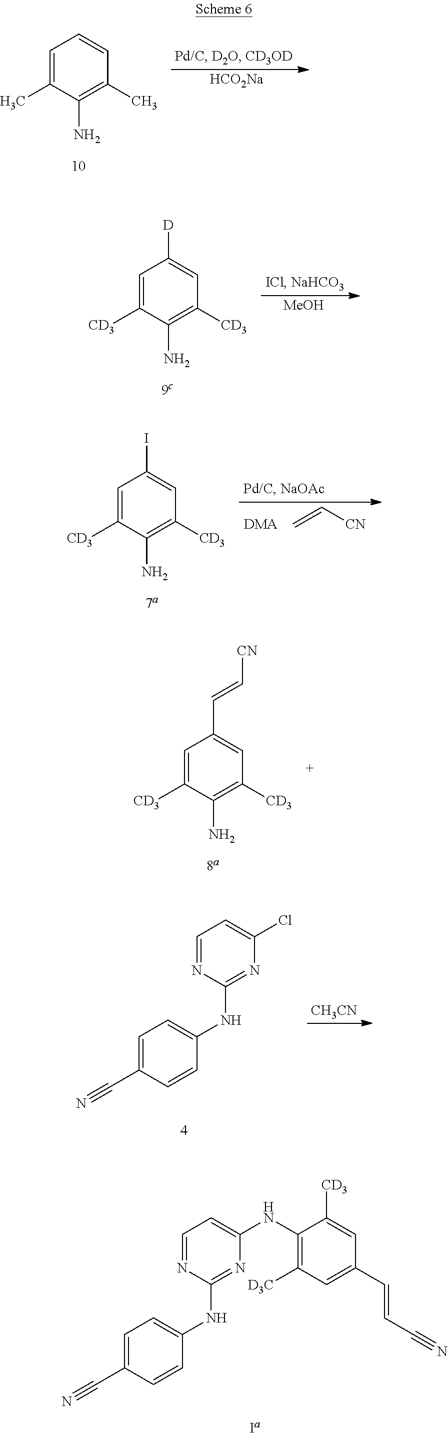

The above synthetic methods are to serve as demonstration that compounds of formula one are accessible to one skilled in the art using existing synthetic methods while employing deuterium reagents or deuterium containing components. These methods are in no way meant to be limiting or the only means to access compounds of formula I. Other methods are depicted in published patent applications WO1999/50250A1, WO2004/016581A1 and WO2006/125809A1, all of which are hereby incorporated by reference herein. Still other methods involving deuterium/hydrogen exchange may also be of interest optionally in the presence of a transition metal catalyst. Furthermore, several additional known methods to make useful intermediates such as compounds such as 7, 8 or 9 can be found in the literature using a variety of well known synthetic methods. These will be apparent to one skilled in the art.

### EXAMPLES

### Example 1

### (E)-4-((4-((4-(2-Cyanovinyl)-2,6-dimethylphenyl)amino)pyrimidin-2-yl)amino)benzonitrile-d₆ (Compound D)

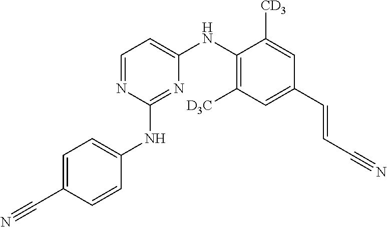

### (a) 2,6-Dimethylaniline-d₇

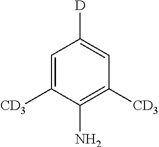

A mixture of 2,6-dimethylaniline (1.2 g, 10 mmol) in CD₃OD (1.5 mL) and D₂O (1.5 mL) was treated with Pd/C (200 mg), sodium formate (100 mg) and heated at 160° C. overnight resulting in a pink solution. The mixture was cooled and filtered using methanol. The solvents were removed in vacuo to provide the desired product (700 mg, 58%) which was used without further purification. It is noted that incomplete D incorporation was observed in this case and is shown in the spectral data. In this case the 4-aryl position further complicates the spectral data. All visible lines in the ¹H-NMR spectra are reported. ¹H-NMR (300 MHz, CDCl₃) δ ppm 6.94 (m, 2H), 6.75 (s, 0.26H) (incomplete deuteration at the 4-aryl position), 3.53 (br s, 2H), 2.21 (m, 0.9H) (incomplete deuteration at the 2,6-methyl position). LCMS (m/z) ES+ 129 (M+1).

### (b) 2,6-Dimethyl-4-iodoaniline-d₆

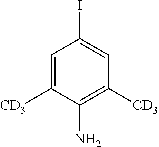

A suspension of 2,6-dimethylaniline-d₇ from step 1a (700 mg, 5.4 mmol) in methanol (20 mL) was treated with sodium bicarbonate (1.4 g, 16.3 mmol) and a mixture of ICI (970 mg, 6.0 mmol) in dichloromethane (6 mL) was added over 10 minutes. The resulting mixture was stirred for 30 minutes at which time TLC indicated the reaction was complete. The solids were removed by filtration and the mixture was concentrated in vacuo to provide a dark oil (1.5 g, 99%) which was used in the next step without further purification. It is noted that incomplete D incorporation in the previous step was carried through into the product from this step. All visible lines in the ¹H-NMR spectra are reported. ¹H-NMR (300 MHz, CDCl₃) δ ppm 6.93 (m, 2H), 3.60 (m, 2H), 2.03 (m, 0.4H) (incomplete deuteration at the 2,6-methyl position from the previous step corresponding to ˜6% protio content). LCMS (m/z) ES+254 (M+1).

### (c) (E)-3-(4-Amino-3,5-dimethylphenyl)acrylonitrile-d₆

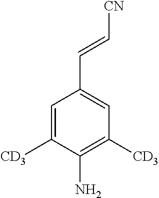

A mixture of 2,6-dimethyl-4-iodoaniline-d₆ (1.5 g, crude from step 1b), acrylonitrile (477 mg, 9 mmol), Pd/C (50 mg) and sodium acetate (590 mg, 7.2 mmol) were stirred in N,N-dimethylacetamide at 130° C. overnight. Water was added and the aqueous layer was extracted with ether. The organics were dried over sodium sulfate, filtered and concentrated. The residue was purified by flash chromatography on silica gel (4:1 PE/EtOAc) to give a dark oil which was dissolved in EtOH/iPr₂O (2 mL/1 mL). The mixture was heated at 60° C. for 30 min before the addition of 6N HCl in iPrOH (0.25 mL). The mixture was stirred at 60° C. for another 30 min filtered and dried overnight to give (E)-3-(4-amino-3,5-dimethylphenyl)acrylonitrile-d₆ (220 mg, 21%) as a pale yellow solid. It is noted that incomplete D incorporation in the step (a) was carried through into the product from this step. All visible lines in the ¹H-NMR spectra are reported. ¹H-NMR (300 MHz, DMSO-d₆) δ ppm 7.38 (d, 1H), 7.20 (s, 2H), 6.86 (br s, 2H), 6.02 (d, 1H), 2.11 (d, 0.82H) (incomplete deuteration at the 2,6-methyl position corresponding to ˜13%). LCMS (m/z) ES+179 (M+1).

### (d) (E)-4-((4-((4-(2-Cyanovinyl)-2,6-dimethylphenyl)amino)pyrimidin-2-yl)amino)benzonitrile-d₆

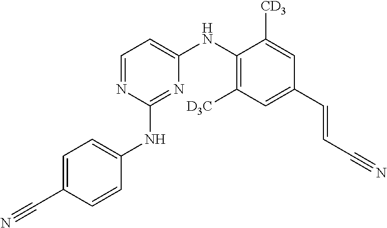

A mixture of (E)-3-(4-amino-3,5-dimethylphenyl)acrylonitrile-d₆ (220 mg, 1 mmol) and 4-((4-chloropyrimidin-2-yl)amino)benzonitrile (prepared according to WO 2004/016581) in acetonitrile were stirred at reflux for 3 days. The mixture was cooled to rt and the solvents evaporated. The mixture was neutralized with potassium carbonate (aq) and extracted with methylene chloride. The organic layer was dried over sodium sulfate, filtered and concentrated. The residue was purified by flash chromatography on silica gel (50:1 DCM:MeOH) to provide (E)-4-((4-((4-(2-Cyanovinyl)-2,6-dimethylphenyl)amino)pyrimidin-2-yl)amino)benzonitrile-d₆ (240 mg, 64%) as a grey solid: ¹H NMR (300 MHz, DMSO-d₆) δ 9.62 (s, 1H), 8.94 (s, 1H), 8.01 (d, 1H), 7.65 (d, 3H), 7.63 (d, 4H), 6.46 (d, 1H) (Note a small signal at 2.13 ppm is also observed in the final product consistent with the above intermediates showing less than complete deuterium incorporation at the 2,6-dimethyl groups which originated from step (a) above. ES+MS: 373.2 (M+1).

### BIOLOGICAL ASSAYS

### Inhibition of Viral Replication

### Example 2

### Pseudo-Typed HIV Antiviral Assay (PHIV Assay)

Overview of PHIV Assay. Pseudo HIV is an HIV-based vector system. The vector incorporates a number of safety features to prevent replication and to inhibit the pathogenicity of the virus. The vector also incorporates a luciferase reporter gene for easy readout. With this system, events of the HIV life cycle after entry and through integration can be easily assayed in a BSL-2 environment. The screen will detect inhibitors of uncoating, reverse transcription, RNase H, and integration.

Cells. The assay is typically run using CIP4 and CIP4-Luci cells in parallel. CIP4 cells are derived from 293 cells which are human embryonal kidney cells transformed with sheared Adenovirus type 5 DNA. 293T cells have been modified by transfection of SV40 T antigen. 293 TAg/hsr-A pCIP4 or “CIP4” cells have been further modified by transfection with a macrophage attachment factor to improve adherence to plastic. The CIP4 cells can be acutely infected with pseudotyped HIV virus and are used to measure the antiviral response. CIP4-Luci cells are chronically infected with pseudotyped HIV virus (i.e. they carry the integrated provirus) and are used to measure non-specific toxicity. Both cell lines are maintained in DMEM/F12 (Invitrogen cat. No. 13330-032) medium with 10% FBS.

Pseudotyped HIV. PHIV is a VSV-G pseudotyped, replication defective HIV vector that expresses a luciferase reporter. The PHIV assay detects HIV inhibitors acting after entry and through integration (e.g. uncoating, RT, RNase H, and integrase inhibitors). It will not detect HIV entry inhibitors, or inhibitors acting from gene expression through virus maturation (e.g. CCR5, tat, rev, or protease inhibitors). An HIV IIIB vector was constructed with tat, vif, vpu, vpr, nef, and env deletions. A CMV promotor replaces 5′ LTR U3 and a deletion within 3′ LTR U3 exists. A luciferase reporter is incorporated and is driven by the SFFV promoter. The construct is packaged by co-transfecting with a VSVg expression vector (FIG. 1).

Compound Titration. Antiviral compounds are diluted to 5 mM in 100% DMSO. Starting at 5 mM, the compounds are serially diluted by a factor of 4 across 10 wells of a 96 well plate. Two ul from each well is spotted onto each of 4 assay plates. The final assay volume is 200 ul, so the final concentration in the first well of the plate is 50 uM. Pseudovirus infected cells are plated at a concentration of 2×10⁴ cells per well. Typically, assays are run on duplicate assay plates. The plates are incubated at 37° C., 5% CO₂ for 48 hours, the medium is aspirated and SteadyGlo luciferase reagent (Promega, catalog number E2550) is added. Luminescense is read in a Packard Topcount plate reader, and a dose response curve is generated.

Compound D has an IC₅₀=0.3 nM in the PHIV assay.

### Formulation Examples

The term “active ingredient” means a compound of the present invention, a tautomer thereof, a pharmaceutically acceptable thereof, or a solvate or hydrate thereof.

### Formulation Example 1

A hard gelatin capsule is prepared using the following ingredients:

| | dose |
|---|---|
| | (mg/capsule) |
| Active ingredient | 250 |
| Starch (dried) | 200 |
| Magnesium stearate | 10 |
| Total | 460 mg |

### Formulation Example 2

A hard gelatin capsule is prepared using the following ingredients:

| | dose |
|---|---|
| | (mg/capsule) |
| Active ingredient | 100 |
| Starch (dried) | 350 |
| Magnesium stearate | 10 |
| Total | 460 mg |

### Formulation Example 3

A hard gelatin capsule is prepared using the following ingredients:

| | dose |
|---|---|
| | (mg/capsule) |
| Active ingredient | 50 |
| Starch (dried) | 400 |
| Magnesium stearate | 10 |
| Total | 460 mg |

### Formulation Example 4

A hard gelatin capsule is prepared using the following ingredients:

| | dose |
|---|---|
| | (mg/capsule) |
| Active ingredient | 5 |
| Starch (dried) | 445 |
| Magnesium stearate | 10 |
| Total | 460 mg |

### Formulation Example 5

A tablet is prepared using the following ingredients:

| | dose |
|---|---|
| | (mg/tablet) |
| Active ingredient | 250 |
| Cellulose (microcrystalline) | 400 |
| Silicon dioxide (fumed) | 10 |
| Stearic acid | 5 |
| Total | 665 mg |

### Formulation Example 6

A tablet is prepared using the following ingredients:

| | dose |
|---|---|
| | (mg/tablet) |
| Active ingredient | 100 |
| Cellulose (microcrystalline) | 550 |
| Silicon dioxide (fumed) | 10 |
| Stearic acid | 5 |
| Total | 665 mg |

### Formulation Example 7

A tablet is prepared using the following ingredients:

| | dose |
|---|---|
| | (mg/tablet) |
| Active ingredient | 50 |
| Cellulose (microcrystalline) | 600 |
| Silicon dioxide (fumed) | 10 |
| Stearic acid | 5 |
| Total | 665 mg |

### Formulation Example 8

A tablet is prepared using the following ingredients:

| | dose |
|---|---|
| | (mg/tablet) |
| Active ingredient | 5 |
| Cellulose (microcrystalline) | 400 |
| Silicon dioxide (fumed) | 10 |
| Stearic acid | 5 |
| Total | 665 mg |

Ingredients are mixed, and compressed to obtain tablets, each weighing 665 mg.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A compound of Formula (I): 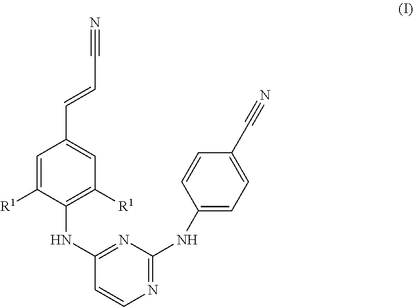
wherein:
each R¹ is independently selected from -D or —H, where at least one R¹ substituent is -D;
or a salt, solvate or hydrate thereof.

2. A compound of Formula (I) 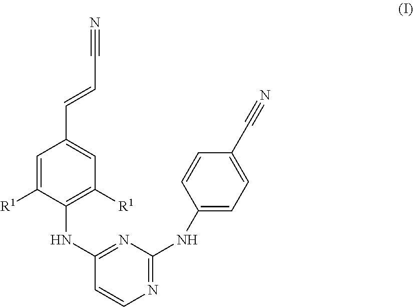
wherein:
each R¹ is independently selected from -D or —H, wherein the level of deuterium incorporation in each R¹ group substituted with -D is at least 50%;
or a salt, solvate or hydrate thereof.

3. A compound of Formula (I) 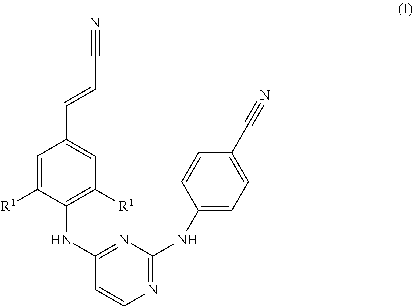
wherein:
each R¹ is independently selected from -D or —H, wherein the level of deuterium incorporation in each R¹ group substituted with -D is at least 70%;
or a salt, solvate or hydrate thereof.

4. A pharmaceutical composition comprising a therapeutically effective amount of a compound of formula (I) or a pharmaceutically acceptable salt, solvate or hydrate thereof and one or more of pharmaceutically acceptable carriers, diluents and excipients.

5. A method of treating a virus susceptible to HIV reverse transcriptase inhibition in a mammal, comprising administering to said mammal a therapeutically effective amount of a compound of formula (I) or a pharmaceutically acceptable salt, solvate or hydrate thereof.

6. A method of inhibiting HIV-related reverse transcriptase in a mammal, comprising administering to said mammal a therapeutically effective amount of a compound of formula (I) or a pharmaceutically acceptable salt, solvate or hydrate thereof.

7. A method of treating reverse transcriptase inhibitor-resistant HIV in a mammal, comprising administering to said mammal a therapeutically effective amount of a compound of formula (I) or a pharmaceutically acceptable salt, solvate or hydrate thereof, wherein the reverse transcriptase inhibitor against which HIV resistance is treated is not a chemically or enzymatically-deuterated reverse transcriptase inhibitor.

8. A method of preventing development of reverse transcriptase inhibitor-resistant HIV in a mammal, comprising administering to said mammal a therapeutically effective amount of a compound of formula (I) or a pharmaceutically acceptable salt, solvate or hydrate thereof, wherein the reverse transcriptase inhibitor against which HIV resistance is prevented is not a chemically or enzymatically-deuterated reverse transcriptase inhibitor.

9. A method of treating a virus susceptible to HIV reverse transcriptase inhibition in a mammal comprising: administering to said mammal a long-acting parenteral composition, wherein the long-acting parenteral composition comprises a therapeutically effective amount of compound as claimed in any of claims 1-4, or a pharmaceutically acceptable salt, solvate or hydrate thereof.

10. A method according to claim 9, wherein the long-acting parenteral composition further comprises at least one additional agent useful in treating HIV infection.

11. A method according to claim 9, wherein the long-acting parenteral composition further comprises at least one additional agent useful as a pharmacological enhancer.

12. A method of treating reverse transcriptase inhibitor-resistant HIV in a mammal comprising: administering to said mammal a long-acting parenteral composition, wherein the long-acting parenteral composition comprises a therapeutically effective amount of compound as claimed in any of claims 1-4, or a pharmaceutically acceptable salt, solvate or hydrate thereof.

13. A method according to claim 12, wherein the long-acting parenteral composition further comprises at least one additional agent useful in treating HIV infection.

14. A method according to claim 13, wherein the long-acting parenteral composition and at least one additional agent exhibit synergism in treating the reverse transcriptase inhibitor-resistant HIV in the mammal.

15. A method according to claim 12, wherein the long-acting parenteral composition further comprises at least one additional agent useful as a pharmacological enhancer.

16. A method of inhibiting HIV-related reverse transcriptase in a mammal comprising: administering to said mammal a long-acting parenteral composition, wherein the long-acting parenteral composition comprises a therapeutically effective amount of a compound as claimed in any of claims 1-4, or a pharmaceutically acceptable salt, solvate or hydrate thereof.

17. A method according to claim 16, wherein the long-acting parenteral composition further comprises at least one additional agent useful in inhibiting HIV-related reverse transcriptase.

18. A method according to claim 17, wherein the long-acting parenteral composition and at least one additional agent exhibit synergism in inhibiting the HIV-related reverse transcriptase in the mammal.

19. A method according to claim 16, wherein the long-acting parenteral composition further comprises at least one additional agent useful as a pharmacological enhancer.

20. A method as claimed in claim 5, comprising further administering at least one additional agent useful in treating HIV infection.

21. A method as claimed in claim 5, comprising further administering at least one additional agent useful as a pharmacological enhancer.

22. A method as claimed in claim 21, comprising further administering at least one additional agent useful as a pharmacological enhancer.

23. A method as claimed in claim 6, comprising further administering at least one additional agent useful in treating HIV infection.

24. A method as claimed in claim 6, comprising further administering at least one additional agent useful as a pharmacological enhancer in treating HIV infection.

25. A method as claimed in claim 24, comprising further administering at least one additional agent useful as a pharmacological enhancer.

26. A method as claimed in claim 7, comprising further administering at least one additional agent useful in treating HIV infection.

27. A method as claimed in claim 7, comprising further administering at least one additional agent useful as a pharmacological enhancer in treating HIV infection.

28. A method as claimed in claim 27, comprising further administering at least one additional agent useful as a pharmacological enhancer.

29. A method as claimed in claim 8, comprising further administering at least one additional agent useful in treating HIV infection.

30. A method as claimed in claim 8, comprising further administering at least one additional agent useful as a pharmacological enhancer in treating HIV infection.

31. A method as claimed in claim 30, comprising further administering at least one additional agent useful as a pharmacological enhancer.

32. A compound selected from the group consisting of:
4-([4-([4-([1E]-2-cyanoethenyl)-2,6-dimethylphenyl]-amino)-2-pyrimidinyl]-amino)-benzonitrile-d₃,
whose structure is: 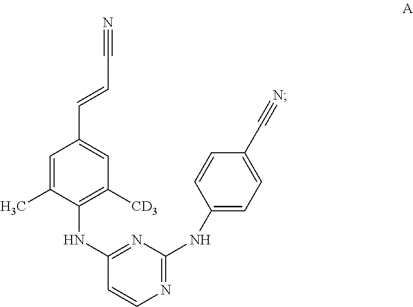
4-([4-([4-([1E]-2-cyanoethenyl)-2,6-dimethylphenyl]-amino)-2-pyrimidinyl]-amino)-benzonitrile-d₄,
whose structure is: 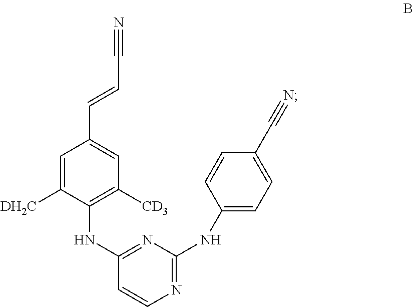
4-([4-([4-([1E]-2-cyanoethenyl)-2,6-dimethylphenyl]-amino)-2-pyrimidinyl]-amino)-benzonitrile-d₅,
whose structure is: 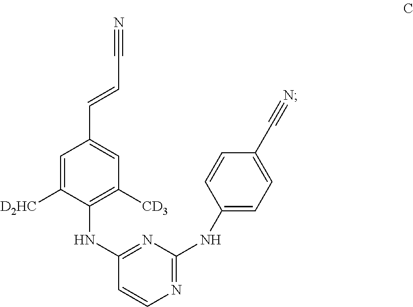
4-([4-([4-([1E]-2-cyanoethenyl)-2,6-dimethylphenyl]-amino)-2-pyrimidinyl]-amino)-benzonitrile-d₆,
whose structure is: 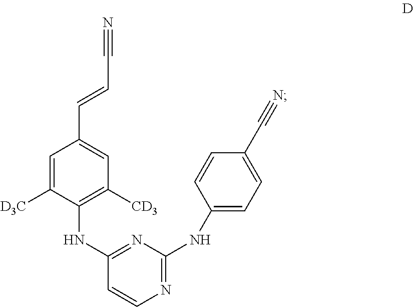
4-([4-([4-([1E]-2-cyanoethenyl)-2,6-dimethylphenyl]-amino)-2-pyrimidinyl]-amino)-benzonitrile-d₂,
whose structure is: 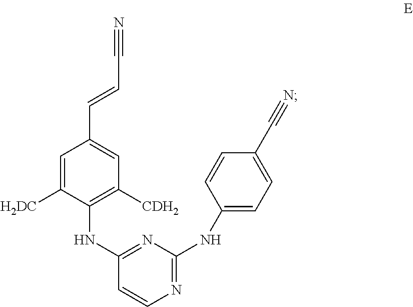
4-([4-([4-([1E]-2-cyanoethenyl)-2,6-dimethylphenyl]-amino)-2-pyrimidinyl]-amino)-benzonitrile-d₃,
whose structure is: 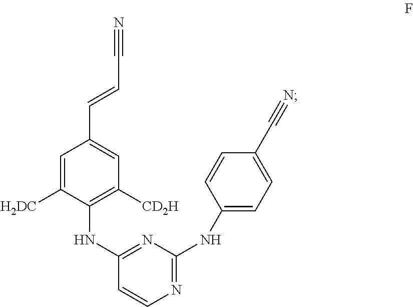
4-([4-([4-([1E]-2-cyanoethenyl)-2,6-dimethylphenyl]-amino)-2-pyrimidinyl]-amino)-benzonitrile-dₐ,
whose structure is: 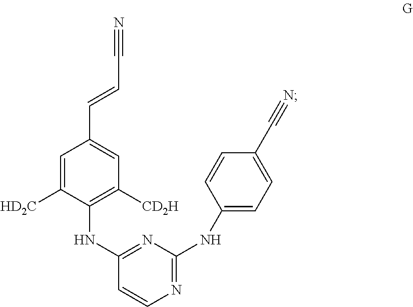
4-([4-([4-([1E]-2-cyanoethenyl)-2,6-dimethylphenyl]-amino)-2-pyrimidinyl]-amino)-benzonitrile-d₂,
whose structure is: 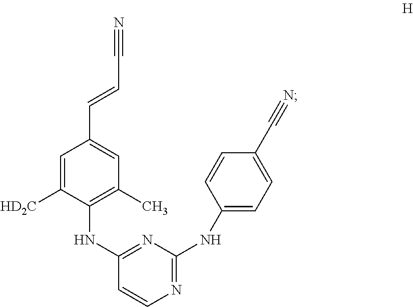
and
4-([4-([4-([1E]-2-cyanoethenyl)-2,6-dimethylphenyl]-amino)-2-pyrimidinyl]-amino)-benzonitrile-d₁,
whose structure is:

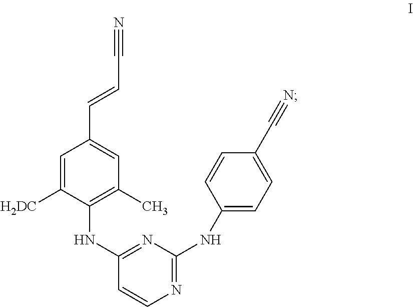

33. A compound which is 4-([4-([4-([1E]-2-cyanoethenyl)-2,6-dimethylphenyl]-amino)-2-pyrimidinyl]-amino)-benzonitrile-d₆.

34. A compound according to claim 33, whose structure is: [chemical structure]
